(19) United States
(12) Patent Application Publication
(10) Pub. No.: US 2017/0251987 A1
(43) Pub. Date: Sep. 7, 2017

(54) System for Measuring and Managing Stress Using Generative Feedback

(71) Applicant: Ronda Collier, Los Gatos, CA (US)

(72) Inventor: Ronda Collier, Los Gatos, CA (US)

(21) Appl. No.: 15/061,666

(22) Filed: Mar. 4, 2016

(51) Int. Cl. A61B 5/00; A61B 5/024; A61B 5/0245; A61B 5/16

(52) U.S. Cl. CPC A61B 5/7405; A61B 5/165; A61B 5/02405; A61B 5/746; A61B 5/7264; A61B 5/0245; A61B 5/6804

(57) ABSTRACT

A computer program product for processing heart rate information signals, which, when run on a computer controls the computer to estimate stress levels of a user in real time and provide generative feedback and alerts to the user when appropriate.

### BACKGROUND OF THE INVENTION

Today many sophisticated diagnoses can be made from vital signs, such as heart beat data, via complex mathematical data analysis techniques. Heart beat data from measurement devices such as ECG and athletic chest strap heart rate monitors is collected and processed to determine metrics such as heart rate and heart rate variability (HRV). This processing expands the range of this data for insight into systemic health and fitness as HRV is a “view” into the Autonomic Nervous System (ANS). The sympathetic “Fight or Flight” branch of the ANS speeds the heart up, while the parasympathetic “rest and digest” branch slows the heart down. The interplay between these two branches of the ANS causes variability in the beat to beat heart rhythm and is a major contributor to HRV. Because our blood pressure, digestion and respiration as well as our thoughts, emotions, perceptions and environment are tightly coupled with the ANS, much can be revealed by monitoring the ANS activity via HRV.

The “Fight or Flight” response is associated with stress. When a perceived threat is encountered, adrenaline causes the heart rate and blood pressure to increase, cortisol is released altering the immune system and suppressing the digestive system, and the brain is affected in the regions that control mood, motivation and fear. In nature, this response turns off once the perceived threat has passed and the body returns to normal. Modern living has provided a near constant supply of “perceived” threats and the Fight or Flight response stays on. Because the brain filters out the familiar even if it is dysfunctional, this chronic Fight or Flight response goes unnoticed, increasing the risk of numerous health problems.

HRV is an established non-invasive and inexpensive method for monitoring the ANS. The standard HRV measures include a variety of time domain, frequency domain and non-linear measures of the heart rate time series. While theses established standard HRV metrics provide insight into general health, they are limited as a on their own and require significant processing in order to be a useful tool for individuals to actively manage daily stressors.

Currently there are applications in this field that measure HRV and provide biofeedback in order to effect a specific physiological change such as breathing frequency or depth. Examples of such products are ‘emWave’ by HeartMath, ‘Heart Tracker’ by Biocom Technologies, ‘Journey to Wild Divine’ by Wild Divine and ‘Stress Doctor’ by Azumio. These products are measuring and encouraging a state of “coherence” where the breathing rate and heart rate are in sync and the ANS exhibits a very specific pattern with activity isolated around the 0.1 Hz HRV frequency. In addition there are health assessment systems, such as ‘Heart Rhythm Scanner’ by Biocom Technologies and Nevrokard that measure HRV and provide comprehensive assessment of the ANS.

While the existing HRV products work well while sitting stationary, most use optical methods for collecting the beat to beat intervals. These methods are very sensitive to motion and are not suitable for accurate ANS assessment if the user is moving. In addition they do not provide real time feedback during regular activities as they require the user to pay attention to the application during use.

Many daily stressors are caused by recurring events and thinking patterns, such as heavy traffic and needless worry. Changing our patterns require behavior changes which are notoriously difficult to effect. Where bio-feedback is the process of becoming aware of physiological functions (breathing, muscle tone, skin conductance) and learning to manipulate these functions while engaging with the biofeedback device, generative feedback is the process of becoming aware of behaviors as they happen, and learning to change these behaviors. Real time generative feedback not only brings awareness to recurring behavior patterns as they happen, it also drives behavior changes through real time audio and visual feedback. Identifying and then correcting a single thought or behavior that crops up many times can have a far reaching impact on overall behavior that affects stress levels and health. Thus generative feedback provides essential feedback to identify behaviors, reduce stress levels and improve health.

A common example of generative feedback can be illustrated by considering a hybrid car. These vehicles have an “Energy Monitor” display that shows the driver, in real time, when the gas engine or electric motor is engaged. The driver who keeps an eye on this and wants to stay on the electric motor as much as possible will soon realize that by accelerating more slowly, the car will remain on the electric motor longer. Thus by changing a single behavior (accelerating more slowly) that is repeated over and over, a significant change is affected in the gas engine to electric motor use ratio and gas mileage increases. This same principle is applied herein.

When monitoring stress for extended periods (beyond a five minute spot check), traditional visual feedback methods fall short. By utilizing audio based feedback, the user may go about his/her daily activities without the need to look at an application for visual feedback. In the 1980's the Bone Phone came out. It looked like a long sock that you would drape over your shoulders. It had an FM tuner and when you turned it on it would send sound vibrations to your ear bones through your collar bones. The result is a discrete way to listen to music that nobody else could hear and that freed your hearing to engage in daily activities. This principle can be applied to generative feedback techniques to provide discrete and non-obtrusive feedback with a form factor that integrates into heart rate monitors, ear buds or wrist bands.

As a hypothetical example, consider a single man in his early 30's who is happily married, has no children, a secure job and no debt. He exercises regularly and is generally healthy. Every day, in the same traffic area of his commute he experiences frustration bordering on road rage. In addition, a certain co-worker with whom he must regularly interact causes him anxiety. He also has the habit of misplacing his keys and his sunglasses and becoming very frustrated every time he does this. Little does he know that it is the daily stressors such as traffic, frustrating people, and even hassles like losing keys, that, over time, contribute to heart disease and hypertension as he ages. The main problem is that because these stressors occur on a regular or daily basis, he is completely unaware that his physiology has entered a stressful state. The addition of a stress detection apparatus with generative feedback could bring awareness to the man that he is unconsciously entering a state of Fight or Flight on a regular basis. This simple awareness could allow him to make new choices during his commute and other daily hassles, thus reducing the accumulation of stressors that can contribute to ill health in middle age.

### SUMMARY OF THE INVENTION

A system which addresses the problem of detecting stress in real time during daily activities and alerting the individual in real time is missing in the art. In addition a method wherein the state of the Autonomic Nervous System is measured and related feedback provided to an individual who is engaged in daily activities, with the specific function to assist in reducing stress and effecting behavior in real time, is also missing in the art. Accordingly, the present disclosure is directed to computer program products and methods for algorithmic processing of heart rate data, algorithmic customization via adaptive scaling of the HRV parameters for an individual and further to providing audio and visual alerts as generative feedback to the user based on the processing, when appropriate. The audio feedback may be part of a stress level measurement audio feedback loop, wherein the audio alert changes in response to changes in the state of the ANS. In addition this audio can be generated with the intention of affecting a change in the ANS via resonant frequencies that induce calm. The audio feedback may be a traditional speaker or it may be a bone conduction speaker providing discrete interaction. In addition, the algorithm may be re-programmed on an individual computer platform and customized to a specific user.

In one embodiment of a system disclosed herein, a tunable stress detection apparatus and a generative feedback delivery mechanism is disclosed. This system consists of a heart rate sensing device, a computing platform such as a PC, mobile phone, tablet, or other processing hardware, an audio feedback component such as a traditional audio speaker, headset or a bone conducting transducer, which may or may not be embedded in the heart rate sensing device and a visual feedback device which may or may not be embedded in the computing platform. This heart rate sensing device and associated speaker, headset or bone conducting transducer may be in the form of a chest strap, wristband, headphone or many other possible stick on or embedded clothing venues, and may be worn by the user full or part time. This device may communicate, via any two way wired or wireless communication protocol, to any computing platform capable or executing the stress detection technique (disclosed herein) and deliver the desired generative feedback signal.

With reference to the example above, the man's heart rate data can be collected by numerous methods, analyzed on the chosen compute platform, and generative feedback delivered, alerting the man that he is in a physiological state of stress. The generative feedback, which may be and not limited to an audio signal, such as music, a tone, or a series of tones, is delivered each time the man enters a pre-selected ANS state. This same technique can be used to prompt the man to do some deep breathing which is known to affect the ANS. As an example, the note “C” may indicate breathe in, and the note “D” may indicate breath out. The frequency can be modulated in any way, such as a 1-2 count ratio of breath in and breath out, which has been shown to balance the nervous system, or to modulate breathing to 1 Hz, which has shown to help asthma patients. The note or composition of notes may also create resonance in the man that is similar to the resonance one may achieve while singing or chanting. In addition the music may be created from the heart beat pattern of the person's own heart beat when they were happy, or it could be a song of their choosing.

The feedback may also be visual in the form of a graphic displayed on the computing platform. Because the pattern embedded in the heart beat contains chaotic and fractal patterns, the visual may be and not limited to an attractor pattern or fractal that is representative of fractal components of the user's heart rate time series. These patterns may change as the state of the individual changes, thus giving visual feedback of the changing physiological state.

### BRIEF DESCRIPTION OF THE DRAWINGS

In the drawings appended hereto like reference numerals denote like elements between the various drawings. While illustrative, the drawings are not drawn to scale. In the drawings:

FIG. 1 is a system level depiction of the overall communication platforms.

FIG. 2 is a flow chart showing the steps for generating training and test vectors for the MLP.

FIG. 3 is a flow chart showing the steps for acquiring the RR intervals and then calculating the stress and generating alerts.

FIG. 4 is represents the RR interval filter.

FIG. 5 shows the HRV calculations on a 5 minute window of RR intervals.

FIG. 6 is a detailed depiction of the normalization and custom HRV scaling.

FIG. 7 shows the audio feedback flow.

FIG. 8 shows an alternative ANS detection scheme that includes non-linear HRV parameters.

FIG. 9 illustrates a flow chart for generating test and training vectors that include non-linear HRV parameters.

FIG. 10 shows real life HRV, heart rate and stress levels of an individual working and having their computer hang.

FIG. 11 shows HRV, heart rate and stress levels during an acupuncture session.

FIG. 12 shows the application display of heart rate, HRV and stress levels.

FIG. 13 shows an example of visual representations of stress levels.

### DETAILED DESCRIPTION

Abbreviations and Acronyms, Definitions

In the following, we refer to various quantities with abbreviations as follows:

Generative Feedback=Feedback that tracks behavior and also drives behavior

ANS—Autonomic Nervous System

ULF=Ultra low frequency

VLF=very low frequency

LF=low frequency

HF=high frequency

HR=heart rate

HRV=heart rate variability

PSD=Power Spectral Density

rMSSD=Root-mean-square of the successive normal sinus RR interval difference

SDNN=standard deviation of all normal sinus RR intervals

RR Interval=Time duration between two consecutive R waves of the ECG.

Ectopic Beat=An irregular beat arising in the heart due to variations in the hearts electrical conductance system

R wave=The first upward deflection in the ECG waveform

ECG=Electro Cardiogram used to monitor heart electrical activity

EEG=Electroencephalogram measures of brain electrical activity

ApEn=Approximate Entropy which quantifies the regularity of RR intervals

DFA=Detrended Fluctuation Analysis permits detection of self similarity in RR intervals

FD=Fractal dimension is a measure of regularity of RR intervals and quantifies sensitivity to initial conditions.

LLE=Local Lyapunov Exponent is a measure of chaoticity of RR intervals

Poincare'Plot=Graphical representation of short term and long term HRV

Holter Monitor=A portable device for recording heartbeats over a period of 24 hours or more.

System Overview

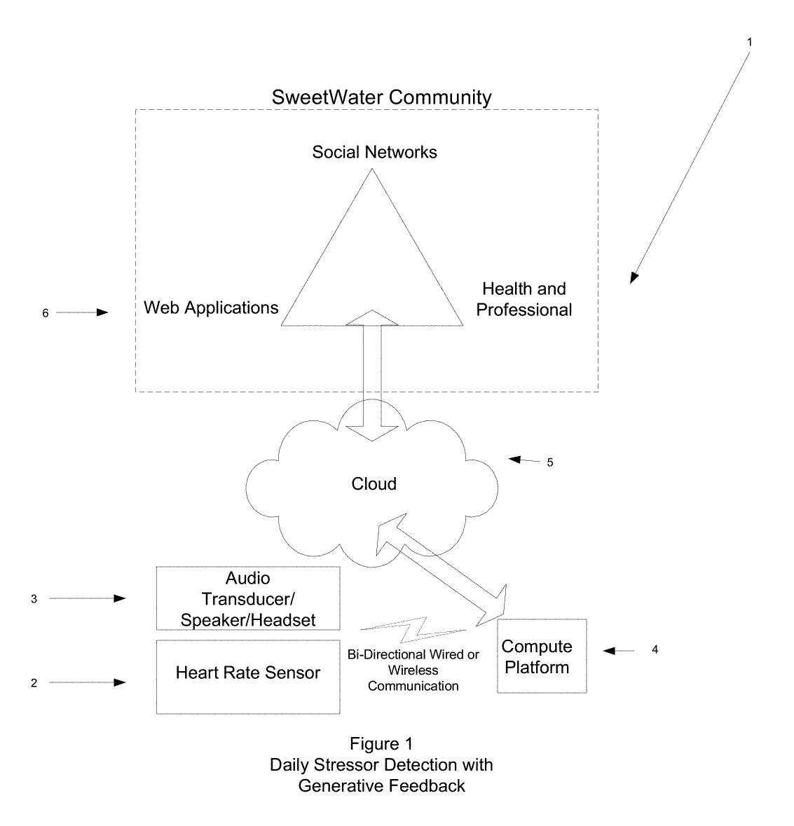

With reference first to FIG. 1, the present disclosure is directed to a system 1 and methods, described further below, for monitoring user heart rate for analysis to detect daily stress that causes imbalance to the Autonomic Nervous System. The present disclosure is directed to analysis of that data on the computer platform or in the cloud, and further to providing ongoing real time feedback and alerts in the form of audio, video, alphanumerical or graphical media. The audio feedback may be transmitted wirelessly or via wire to a bone conducting transducer, speaker or headset 3 worn by an individual.

Monitoring of heart rate is accomplished via a medical or consumer heart rate measurement apparatus including and not limited to an ECG, Holter Monitor, chest strap, optical, or clothing incorporated sensor 2. This heart rate data is transmitted via wire or wireless to a computing platform 4 for analysis. The computing platform includes and is not limited to a smart phone, tablet or desktop computer.

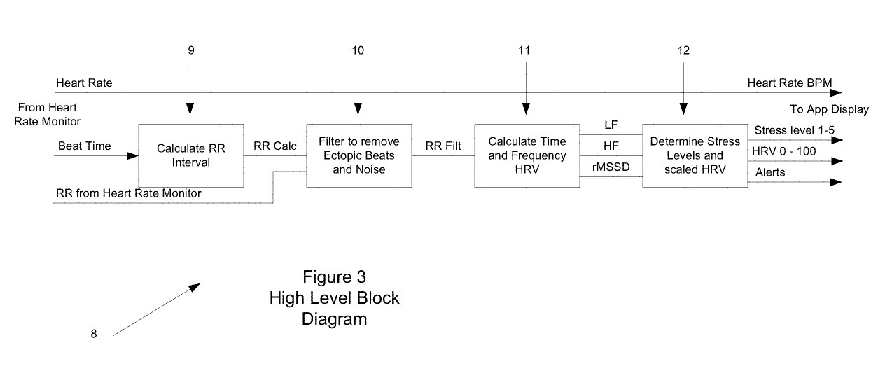

Referring to FIG. 3, the beat to beat or RR intervals are then calculated 9 from the heart rate data if they are not provided directly from the heart rate measurement device. These intervals are filtered 10 and then processed to calculate the corresponding HRV values 11. The heart rate and HRV information are input into a stress level detection algorithm 12 to classify the data into one of five stress levels.

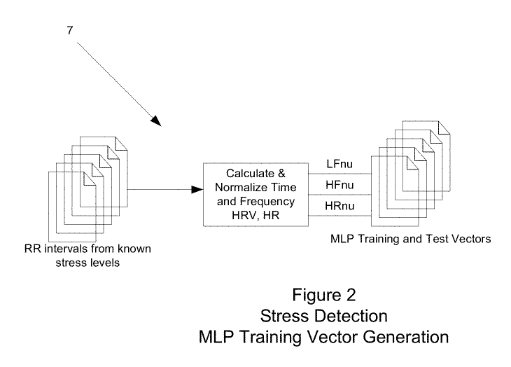

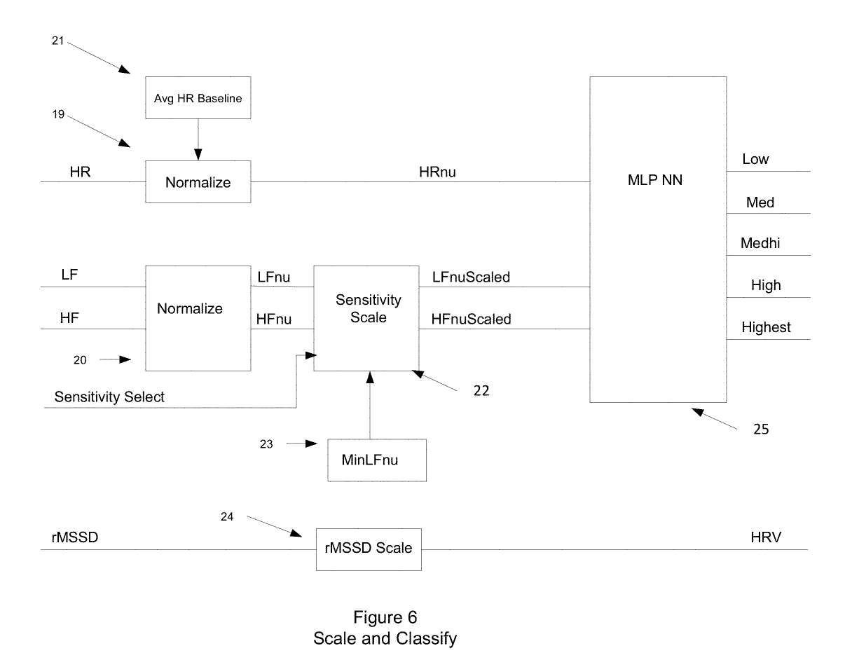

FIG. 6 details the stress level detection algorithm. The HRV parameters LF and HF are normalized and then scaled and, along with the normalized heart rate HRnu, providing the inputs to the Multilayer Perceptron Neural Network (MLP). The original MLP training vectors were calculated from a set of test vectors associated with known stress states as shown in FIG. 2.

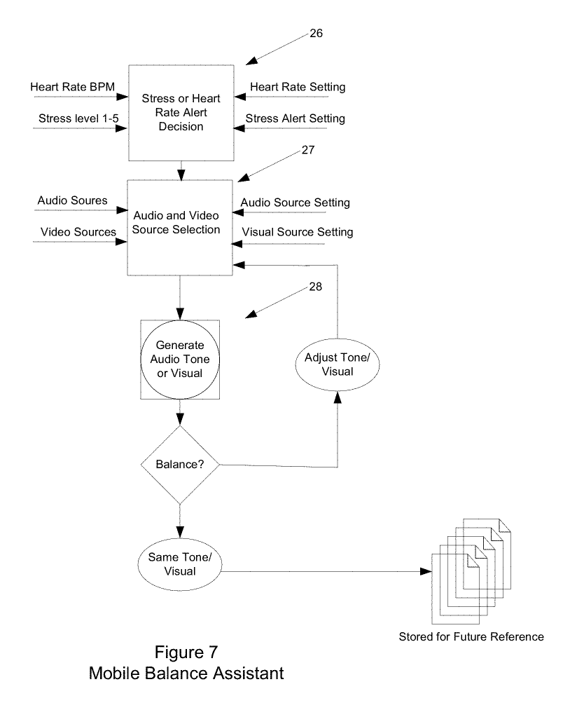

FIG. 7 illustrates the alert detection flow. When a user specified stress level is detected 26 an alert signal is received by the alert source detection module 27. If the alert is audio, it is sounded from the compute platform or transmitted to the user worn bone transducer, headset or speaker. If the alert is visual, it is displayed on the compute platform. As the program continues to detect stress levels, the audio and/or visual alert 28 is adjusted. This adjustment can be a result of a change in stress levels or it can be a result of no change in stress levels with the intention of inducing a lower stress state in the user. This feedback loop consists of tone generation or visual indicator->HRV measurement->tone/visual adjustment->tone/visual generation. This iterative process may continue until the desired outcome is achieved. The alert details and associated stress levels are stored for future use.

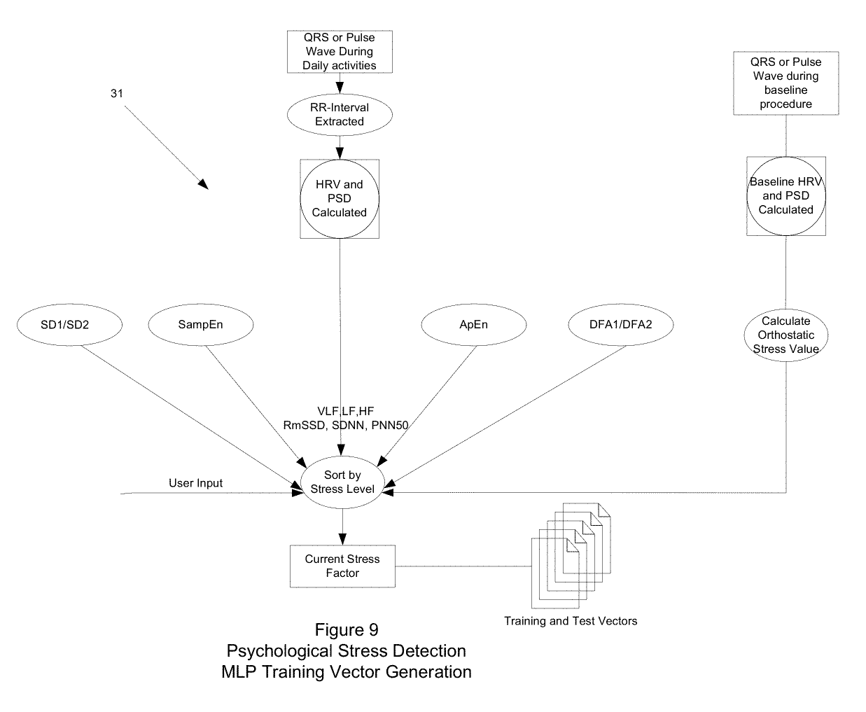

Referring again to FIG. 1, at the end of a monitoring session, details of the session, including the raw RR intervals are stored and uploaded to the cloud to be used in the web applications. In addition the raw data from an individual, combined with user input, is used to create custom training vectors for the MLP 25, (FIG. 6) as shown in FIG. 9. The hidden node weights from the custom classifier (MLP) are then downloaded to the compute platform and a new individually customized stress detection algorithm is used for future monitoring sessions. This process can be repeated indefinitely.

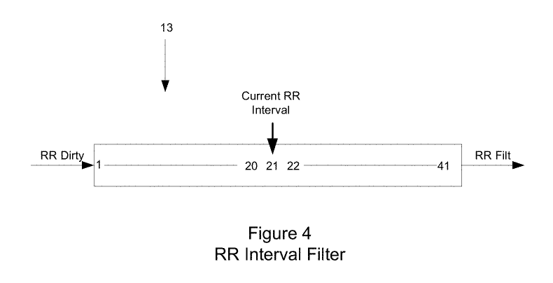

Referring again to FIG. 3, the heart rate monitor may provide the heart beat time or the RR intervals directly. In the event that the beat time is provided, the RR intervals are calculated as RRt=Beat Time (t+1)—Beat Time (t). The RR intervals, whether they were calculated or provided by the heart rate monitor, are then filtered 10 to remove any noise or ectopic beats. FIG. 4 shows the detailed filter 13 that works as follows:

41 RR intervals are queued in a “FIFO” type array

The 21^{st} RR interval is the current intervals

Intervals 1-20 and 22-41 are averaged

If the current interval is +1-20% of the averages of 1-20 and 22-41 then it is considered a normal and labeled “N”.

If the current interval falls outside the +1-20% range it is labeled “O”

If an interval is less than 0.4 seconds it is labeled “I”

If an interval is more than 2 sec it is labeled ‘X”

Only “N” intervals are used for HRV calculation

This is repeated each time a new RR interval is input into the FIFO

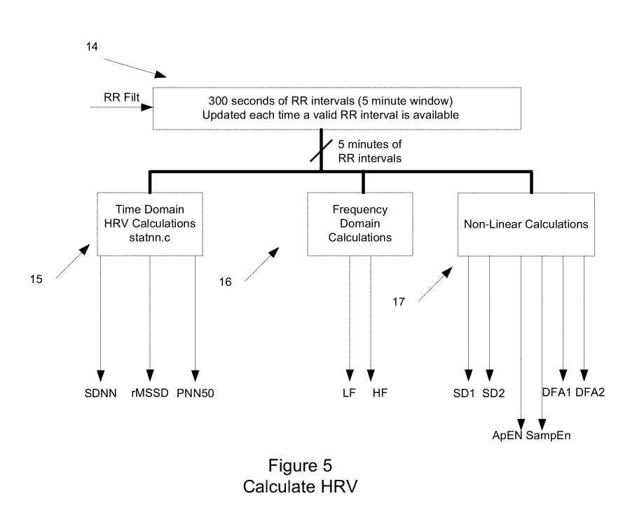

The filtered RR intervals are stored in another “FIFO” type array (FIG. 5) 14, and 300 seconds worth of RR intervals are collected to create a 5 minute window that is then processed. The time domain HRV calculation block 15 computes and is not limited to rMSSD, SDNN and PNN50. The frequency domain HRV calculation block 16 computes and is not limited to LF and HF, The Non-Linear calculations block 17 computes and is not limited to SD1/SD2, ApEN, SampEn, DFA1/DFA2.

Once the time and frequency HRV parameters are calculated, they are processed to determine the stress level of the individual. FIG. 6 shows one such embodiment of the stress detection process. The heart rate, LF and HF values are normalized 19,20 as follows:

Normalize HR 19:

Average HR during baseline is recorded in register Avg_HR_Baseline 21

HRnu=Current HR−Avg_HR_Baseline

Normalize LF, HF 20:

LFnu=LF/(LF+HF)

HFnu=HF/(LF+HF)

Because HRV varies for many reasons, including personal physiology, age, gender and chronic states of the nervous system, (such as chronic stress, anxiety or depression), the LF and HF values, which are highly representative of the activity in the sympathetic branch of the ANS, may be scaled 22, The scaling is crucial for individuals with chronic stress because no matter what they are doing they will always display the highest stress level and will not be able to gain benefit from monitoring. This adaptive scaling allows individuals who have over active sympathetic activity (chronic high stress) to still be able to see stress levels that range from low to high. Note that the method shown here is adaptive to an individual, meaning that the lowest value of an individual's recorded LFnu (LFnu is a major indicator of Fight or Flight) is stored in a register MinLFnu 23 and contributes to the extent of the scaling. Therefore an individual with very high LFnu (higher stress) will get more scaling applied than an individual with a lower LFnu. In this embodiment there are 5 levels of scaling or sensitivity where level 1 provides the most scaling and level 5 provides no scaling. The sensitivity scaling method includes and is not limited to the following:

Sensitivity:

At the end of each session, the following parameter is stored in a register MinLFnu and used for scaling. This allows adaptable scaling for each individual physiology.

a. MinLFnu=Minimum of LFnu of all previous sessions.
i. The default value is 0.6
ii. This register is always updated after the first session
iii. This register will subsequently only be updated if the Min(LFnu) is less than the current register value

Sensitivity Scaling for level 5

No Scaling

Sensitivity scaling for level 4

b. LFnuScaled=LFnu−(MinLFnu−0.73)

c. HFnuScaled=LFnu+(MinLFnu−0.73)

Sensitivity scaling for level 3 (default)

d. LFnuScaled=LFnu−(MinLFnu−0.68)

e. HFnuScaled=LFnu+(MinLFnu−0.68)

Sensitivity scaling for level 2

f. LFnuScaled=LFnu−(MinLFnu−0.52)

g. HFnuScaled=LFnu−(MinLFnu−0.52)

Sensitivity scaling for level 1

h. LFnuScaled=LFnu−(MinLFnu−0.48)

i. HFnuScaled=LFnu+(MinLFnu−0.48)

In order to make rMSSD consumer friendly, it is scaled 24 to a range of 0-100 which is easily understood by most people.

rMSSD Scale:

One such embodiment of scaling rMSSD includes and is not limited to:

HRV=(log(rmssd)−0.3)/(2−0.3)*100

HRV will not exceed 100

The normalized heart rate HRnu, normalized and scaled LF and HF (LFnuScaled, HFuScaled) HRV values are used as the inputs to the stress level classifier 25 that outputs the detected stress level, from low to highest, at the rate of a new value each second.

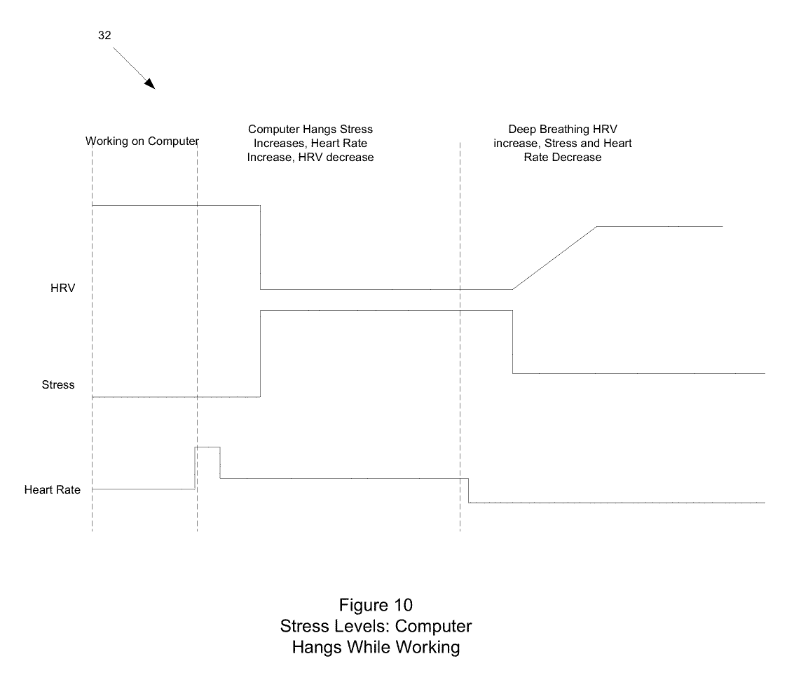

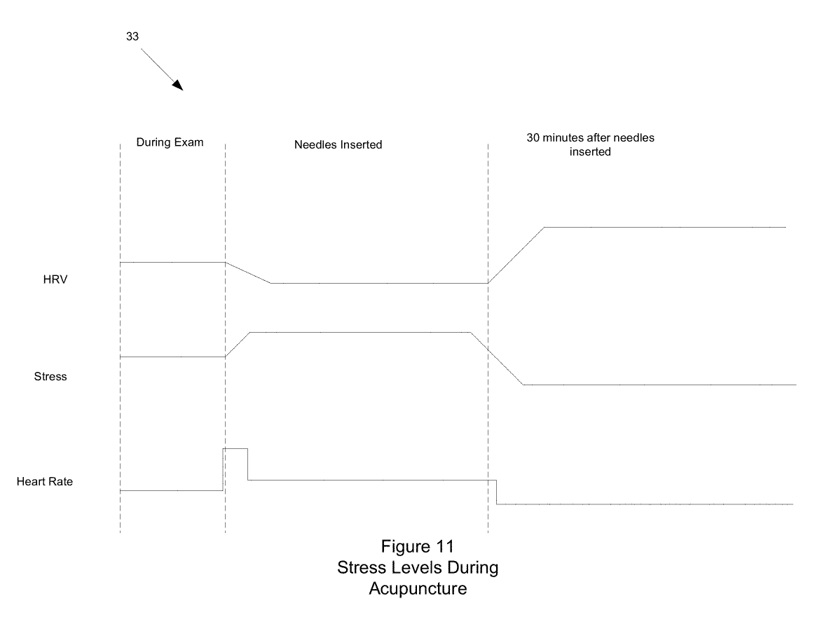

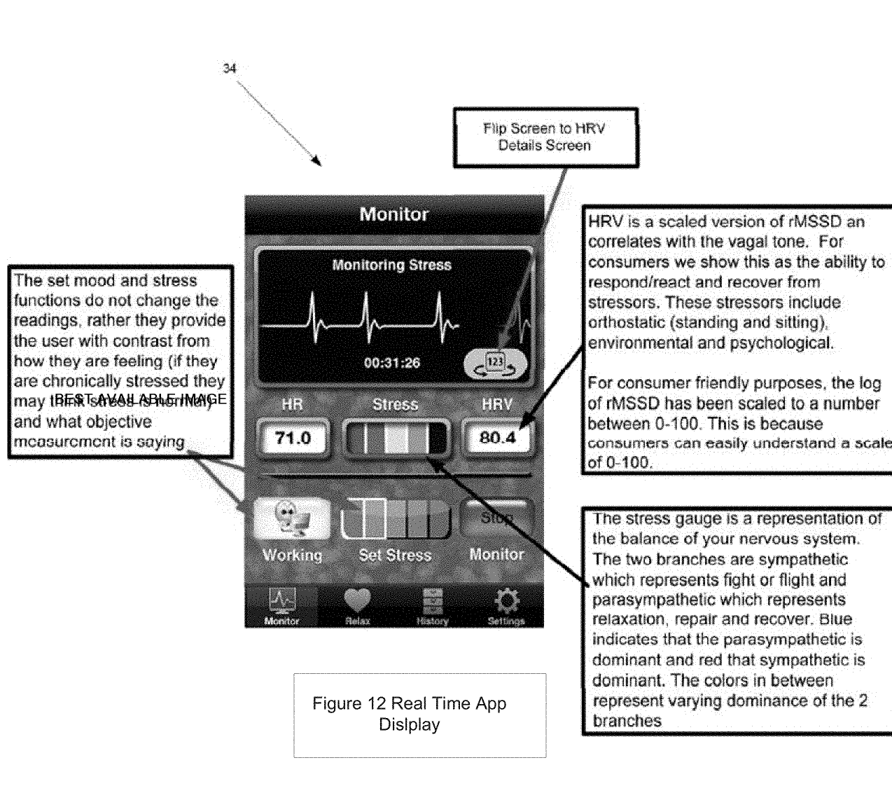

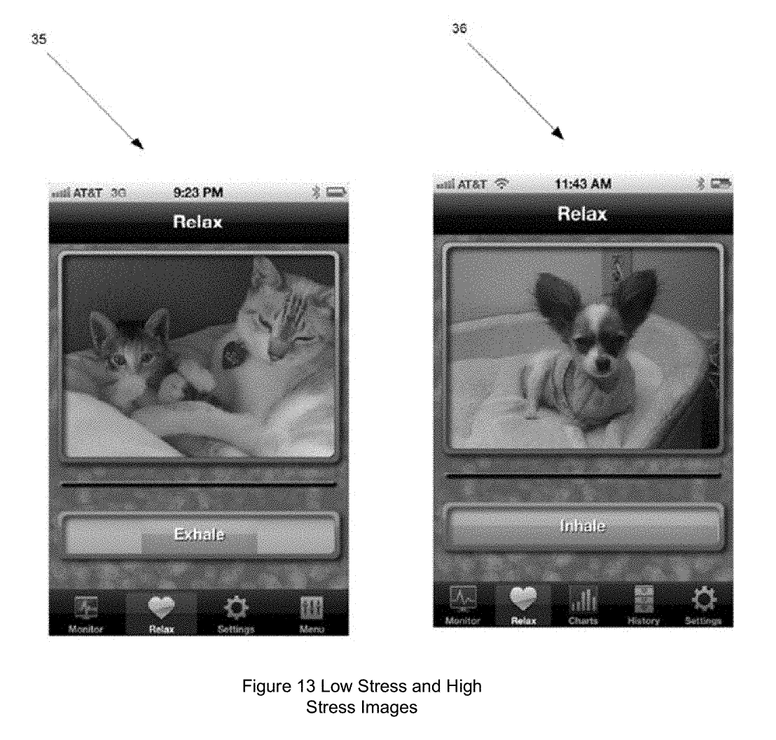

As seen in FIG. 12 the current heart rate, HRV and Stress level are displayed in the application 34 in real time (updated each second) and FIG. 13 shows a visual representation of low 35 and high 36 stress. The real time data, heart rate, HRV and stress levels, are stored and displayed in a graph 32, 33 as shown in FIGS. 10 and 11.

Multilayer Perceptron Detailed Description

The Multilayer Perceptron is a feed forward neural network that maps a set of inputs onto a set of appropriate outputs. The MLP may have and is not limited to the following properties:

1 input layer, 3 input nodes (HRnu, LFnuScaled, HFnuScaled)

I Hidden layer

24 nodes in hidden layer

Sigmoid function

1 output layer, 5 output nodes (Stress Level 1-5)

Referring to FIG. 2 the MLP was initially trained using data taken from volunteers while driving on a prescribed route including city streets and. The drivers were presented with the following route, each invoking a range of stress reactions:

Rest in a garage

Drive busy city streets

Drive on the highway

Enter toll booths

The RR intervals and heart rate for low to high stress states were extracted from the data 7 and the HRV calculations were applied to the RR intervals. The resulting HRV and HR were grouped into low, med, medhi, high and highest stress training and test vectors, and applied to training of the MLP.

Once the initial MLP and alpha version of the app was available, more vectors were generated by running sessions in the application in a variety of low to high stress situations, labeling these sessions and combining them into the associated HRV parameters into low-high stress levels. These vectors were combined with the driving training vectors to create a final training and test set.

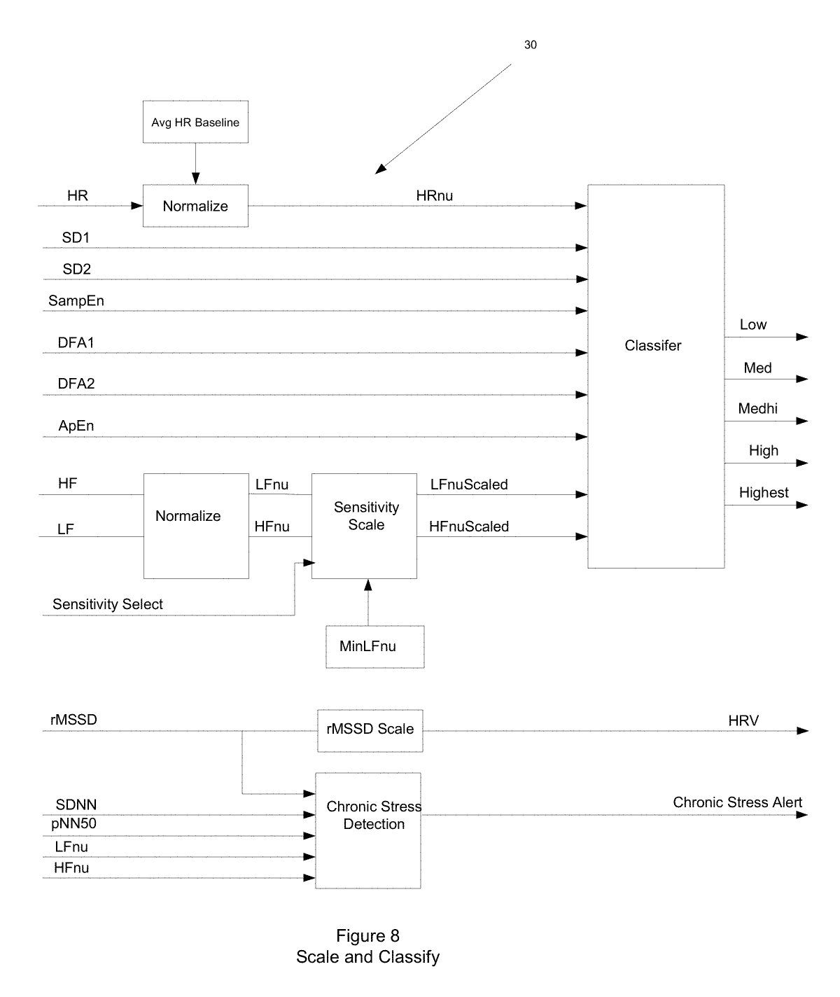

FIG. 8 represent an alternative or complimentary method of stress detection. This method utilizes the non-linear calculations in addition to the time and frequency domain calculations of HRV. Because the RR interval time series of a healthy individual has chaotic and fractal characteristics, the non-linear aspects of HRV can provide deeper insight into the ANS and present an opportunity for early detection and diagnosis for a variety of physical and psychological conditions such as hypertension, heart disease, obstructive sleep apnea, anxiety and depression, to name a few. In addition, burn out or chronic stress can be detected. Tracking these parameters provides individuals and health practitioners a unique insight into the efficacy of treatments.

While a plurality of preferred exemplary embodiments have been presented in the foregoing detailed description, it should be understood that a vast number of variations exist, and these preferred exemplary embodiments are merely representative examples, and are not intended to limit the scope, applicability or configuration of the disclosure in any way. Various of the above-disclosed and other features and functions, or alternative thereof, may be desirably combined into many other different systems or applications. Various presently unforeseen or unanticipated alternatives, modifications variations, or improvements therein or thereon may be subsequently made by those skilled in the art.

Therefore, the foregoing description provides those of ordinary skill in the art with a convenient guide for implementation of the disclosure, and contemplates that various changes in the functions and arrangements of the described embodiments may be made without departing from the spirit and scope of the disclosure. All comparable variations are understood to fall within the framework of the invention as outlined by the following claims.

1. A computer program product that as input receives heart beat information from a heart rate monitor and, when run on a computer:
processes the heart beat intervals to detect the state of the Autonomic Nervous System in real time using a re-programmable personalized Neural Network,
applies adaptive scaling to HRV parameters to customize the stress level detection for an individual,
generates immediate feedback that indicates measured ANS states and, when appropriate, alerts using audio, visual, alphanumeric displays on the computer platform or via a wired or wireless speaker or audio transducer,
generates audio or visual feedback that is composed from the user's own heart rate time series,
generates audio feedback as part of a stress level-audio feedback loop, to a wearable audio transducer or speaker with the intent of affecting a physiological response from the user.
